(19) United States
(12) Patent Application Publication
(10) Pub. No.: US 2026/0017731 A1
(43) Pub. Date: Jan. 15, 2026

(54) GENOMIC PREDICTION METHOD AND APPARATUS BASED ON GENOTYPE-ENVIRONMENT INTERACTION HETEROGENEOUS GRAPH

(71) Applicant: Information Technology Research Center, Beijing Academy of Agriculture and Forestry Sciences, BEIJING (CN)

(72) Inventors: Feng YANG, BEIJING (CN); Kaiyi WANG, BEIJING (CN); Shouhui PAN, BEIJING (CN); Jinlong LI, BEIJING (CN); Dongfeng ZHANG, BEIJING (CN); Zhongqiang LIU, BEIJING (CN); Yanyun HAN, BEIJING (CN)

(21) Appl. No.: 19/012,260

(22) Filed: Jan. 7, 2025

(30) Foreign Application Priority Data
Jul. 11, 2024 (CN) 202410925622.9

(51) Int. Cl. G06Q 50/02; G16B 20/00; G16B 40/20

(52) U.S. Cl. CPC G06Q 50/02; G16B 20/00; G16B 40/20

(57) ABSTRACT

Provided are a genomic prediction method and apparatus based on a genotype-environment interaction heterogeneous graph, relating to the technical field of bioinformatics. The method includes: obtaining genotype data of a to-be-predicted crop variety and generating genotype features of the to-be-predicted crop variety based on the genotype data of the to-be-predicted crop variety; obtaining environmental data of a target environment and generating environmental features of the target environment based on the environmental data of the target environment; generating a heterogeneous graph based on the genotype features of the to-be-predicted crop variety, genotype features of at least one other crop variety, the environmental features of the target environment, environmental features of at least one other environment, and phenotype data; and inputting the heterogeneous graph into a trained heterogeneous graph prediction model to obtain predicted phenotype data of the to-be-predicted crop variety in the target environment outputted by the heterogeneous graph prediction model.

### CROSS REFERENCE TO RELATED APPLICATION

This patent application claims the benefit and priority of Chinese Patent Application No. 2024109256229, filed with the China National Intellectual Property Administration on Jul. 11, 2024, the disclosure of which is incorporated by reference herein in its entirety as part of the present application.

### TECHNICAL FIELD

The present disclosure relates to the technical field of bioinformatics, and in particular, to a genomic prediction method and apparatus based on a genotype-environment interaction heterogeneous graph.

### BACKGROUND

Genomic prediction technology based on genotype data is used to predict and select the phenotypes of breeding populations based on the association between genotype data and phenotypes. This method can shorten the breeding cycle and improve breeding efficiency.

Existing phenotype prediction methods based on genetic data typically rely on linear models, and which overlook the complex interaction between genotypes and environments, resulting in low prediction accuracy. In addition, usually only genomic data is used for genomic prediction, lacking integrated multi omics data such as genomics, transcriptomics, proteomics, and metabolomics.

### SUMMARY

The present disclosure provides a genomic prediction method and apparatus based on a genotype-environment interaction heterogeneous graph, to address the low prediction accuracy of phenotypes in the prior art and achieve improved phenotype prediction accuracy.

The present disclosure provides a genomic prediction method based on a genotype-environment interaction heterogenous graph, including:
obtaining genotype data of a to-be-predicted crop variety and generating genotype features of the to-be-predicted crop variety based on the genotype data of the to-be-predicted crop variety;
obtaining environmental data of a target environment and generating environmental features of the target environment based on the environmental data of the target environment;
generating a heterogeneous graph based on the genotype features of the to-be-predicted crop variety, genotype features of at least one other crop variety, the environmental features of the target environment, environmental features of at least one other environment, and phenotype data, where the heterogeneous graph includes first nodes and second nodes, each first node corresponds to genotype features of one crop variety, and each second node corresponds to environmental features of one environment; a connecting edge between the first nodes reflects a genetic relationship (represented by similarity) between the crop varieties corresponding to the first nodes, a connecting edge between the second nodes reflects a similarity between the environments corresponding to the second nodes, and a connecting edge between the first node and the second node reflects phenotype of the crop variety corresponding to the first node in the environment corresponding to the second node; and
inputting the heterogeneous graph into a trained heterogeneous graph prediction model to obtain predicted phenotype data of the to-be-predicted crop variety in the target environment outputted by the heterogeneous graph prediction model, where the heterogeneous graph prediction model is trained based on a plurality of sets of training data, and each set of training data includes genotype data of a sample crop variety and a phenotype data label of the sample crop variety in a sample environment.

According to the genomic prediction method based on a genotype-environment interaction heterogeneous graph provided by the present disclosure, said generating the genotype features of the to-be-predicted crop variety based on the genotype data of the to-be-predicted crop variety includes:
generating a variety association graph based on the genotype data of the to-be-predicted crop variety and genotype data of a plurality of crop varieties, where each node in the variety association graph corresponds to the genotype data of one crop variety, and a connecting edge between the nodes corresponds to a similarity between the genotype data of the crop varieties; and
inputting the variety association graph into a trained first graph processing model, aggregating genotype data of a first target node with genotype data of other nodes in the variety association graph through the first graph processing model to obtain a first aggregation result, and deriving the genotype features of the to-be-predicted crop variety based on the first aggregation result, where the first target node is a node corresponding to the to-be-predicted crop variety;
said generating the environmental features of the target environment based on the environmental data of the target environment includes:
generating an environmental association graph based on the environmental data of the target environment and environmental data of a plurality of environments, where each node in the environmental association graph corresponds to one piece of environmental data, and a connecting edge between the nodes corresponds to a similarity between the environmental data; and
inputting the environmental association graph into a trained second graph processing model, aggregating environmental data of a second target node with environmental data of other nodes in the environmental association graph through the second graph processing model to obtain a second aggregation result, and deriving the environmental features of the target environment based on the second aggregation result, where the second target node is a node corresponding to the target environment;
where the first graph processing model, the second graph processing model, and the heterogeneous graph prediction model are jointly trained based on the plurality of sets of training data.

According to the genomic prediction method based on a genotype-environment interaction heterogeneous graph provided by the present disclosure, said generating the variety association graph based on the genotype data of the to-be-predicted crop variety and the genotype data of the plurality of crop varieties includes:
obtaining first similarities between the genotype data of the to-be-predicted crop variety and the genotype data of other crop varieties;
determining neighboring crop varieties among the other crop varieties based on the first similarities; and
generating the variety association graph based on the genotype data of the to-be-predicted crop variety and the neighboring crop varieties;
said generating the environmental association graph based on the environmental data of the target environment and the environmental data of the plurality of environments includes:
obtaining second similarities between the environmental data of the target environment and the environmental data of other environments;
determining neighboring environments among the other environments based on the second similarities; and
generating the environmental association graph based on the environmental data of the target environment and the neighboring environments.

According to the genomic prediction method based on a genotype-environment interaction heterogeneous graph provided by the present disclosure, the genotype data can be aggregated with multiple types of single-omics data; and said deriving the genotype features of the to-be-predicted crop variety based on the first aggregation result includes:
obtaining the first aggregation result corresponding to each type of single-omics data, and concatenating all the first aggregation results to obtain the genotype features of the to-be-predicted crop variety;
the environmental data includes multiple types of environmental single-omics data; and said deriving the environmental features of the target environment based on the second aggregation result includes:
obtaining the second aggregation result corresponding to each type of environmental single-omics data, and concatenating all the second aggregation results to obtain the environmental features of the target environment.

According to the genomic prediction method based on a genotype-environment interaction heterogeneous graph provided by the present disclosure, the heterogeneous graph prediction model includes a node feature aggregation module and a phenotype prediction module; and said inputting the heterogeneous graph into the trained heterogeneous graph prediction model to obtain the predicted phenotype data of the to-be-predicted crop variety in the target environment outputted by the heterogeneous graph prediction model includes:
inputting the heterogeneous graph into the node feature aggregation module, aggregating features of a target first node with features of neighboring nodes in the heterogeneous graph through the node feature aggregation module to obtain a first aggregated feature, and aggregating features of a target second node with features of neighboring nodes to obtain a second aggregated feature, where the target first node is a node corresponding to the to-be-predicted crop variety, and the target second node is a node corresponding to the target environment; and
inputting the first aggregated feature and the second aggregated feature into the phenotype prediction module to obtain the predicted phenotype data of the to-be-predicted crop variety in the target environment outputted by the phenotype prediction module.

According to the genomic prediction method based on a genotype-environment interaction heterogeneous graph provided by the present disclosure, said inputting the heterogeneous graph into the node feature aggregation module, aggregating the features of the target first node with the features of the neighboring nodes in the heterogeneous graph through the node feature aggregation module to obtain the first aggregated feature, and aggregating the features of the target second node with the features of the neighboring nodes to obtain the second aggregated feature includes:
by taking the target first node and the target second node as to-be-aggregated nodes, performing the following operations to aggregate the features of the to-be-aggregated nodes with the features of the neighboring nodes:
aggregating the features of the to-be-aggregated node with features of each first neighboring node based on a graph attention mechanism to obtain a first-type feature, where the first neighboring node is the first node among the neighboring nodes of the to-be-aggregated node;
aggregating the features of the to-be-aggregated node with features of each second neighboring node based on the graph attention mechanism to obtain a second-type feature, where the second neighboring node is the second node among the neighboring nodes of the to-be-aggregated node; and concatenating the first-type feature and the second-type feature.

The present disclosure further provides a genomic prediction apparatus based on a genotype-environment interaction heterogeneous graph (referred to as an apparatus for predicting an environmental phenotype of a crop variety), including:
a first feature generation module configured to obtain genotype data of a to-be-predicted crop variety and generate genotype features of the to-be-predicted crop variety based on the genotype data of the to-be-predicted crop variety;
a second feature generation module configured to obtain environmental data of a target environment and generate environmental features of the target environment based on the environmental data of the target environment;
a heterogeneous graph construction module configured to generate a heterogenous graph based on the genotype features of the to-be-predicted crop variety, genotype features of at least one other crop variety, the environmental features of the target environment, environmental features of at least one other environment, and phenotype data, where the heterogeneous graph includes first nodes and second nodes, each first node corresponds to genotype features of one crop variety, and each second node corresponds to environmental features of one environment; a connecting edge between the first nodes reflects a genetic relationship (represented by similarity) between the crop varieties corresponding to the first nodes, a connecting edge between the second nodes reflects a similarity between the environments corresponding to the second nodes, and a connecting edge between the first node and the second node reflects phenotype of the crop variety corresponding to the first node in the environment corresponding to the second node; and
a prediction module configured to input the heterogenous graph into a trained heterogeneous graph prediction model to obtain predicted phenotype data of the to-be-predicted crop variety in the target environment outputted by the heterogeneous graph prediction model, where the heterogeneous graph prediction model is trained based on a plurality of sets of training data, each set of training data includes genotype data of a sample crop variety and a phenotype data label of the sample crop variety in a sample environment.

The present disclosure also provides an electronic device, including a memory, a processor, and a computer program stored in the memory and executable on the processor, where the processor executes the computer program to implement the genomic prediction method based on a genotype-environment interaction heterogeneous graph described above.

The present disclosure further provides a non-transitory computer-readable storage medium that stores a computer program, where the computer program, when executed by a processor, implements the genomic prediction method based on a genotype-environment interaction heterogeneous graph described above.

The present disclosure further provides a computer program product, including a computer program, where the computer program, when executed by a processor, implements the genomic prediction method based on a genotype-environment interaction heterogeneous graph described above.

According to the genomic prediction method and apparatus based on a genotype-environment interaction heterogeneous graph provided by the present disclosure, genotype features are extracted from genotype data of a to-be-predicted crop variety, environmental features are extracted from environmental data of a target environment, and then a heterogeneous graph is generated based on interrelationships between different crops and different environments, as well as known phenotype data of crops in those environments. A trained heterogeneous graph prediction model is used to process the heterogeneous graph to obtain predicted phenotype data of the to-be-predicted crop variety in the target environment. The present disclosure fully considers the relationship between the genotype of the crop variety and the environment when predicting phenotype data, which can improve the accuracy of phenotype predictions.

### BRIEF DESCRIPTION OF THE DRAWINGS

To describe the technical solutions in the present disclosure or in the prior art more clearly, the accompanying drawings required for describing embodiments or the prior art will be briefly described below. Apparently, the accompanying drawings in the following description show some embodiments of the present disclosure, and a person of ordinary skill in the art may still derive other drawings from these accompanying drawings without creative efforts.

FIG. 1 is a flowchart of a genomic prediction method based on a genotype-environment interaction heterogeneous graph according to the present disclosure;

FIG. 2 is a schematic diagram illustrating a heterogeneous graph generation process in the genomic prediction method based on a genotype-environment interaction heterogeneous graph according to the present disclosure;

FIG. 3 is a schematic diagram illustrating a processing process of a heterogeneous graph prediction model in the genomic prediction method based on a genotype-environment interaction heterogeneous graph according to the present disclosure; and

FIG. 4 is a schematic structural diagram of an apparatus for predicting an environmental phenotype of a crop variety according to the present disclosure; and

FIG. 5 is a schematic structural diagram of an electronic device according to the present disclosure.

### DETAILED DESCRIPTION OF THE EMBODIMENTS

To make the objectives, technical solutions and advantages of the present disclosure clearer, the following clearly and completely describes the technical solutions in the present disclosure with reference to the accompanying drawings in the present disclosure. Apparently, the described embodiments are some but not all of the embodiments of the present disclosure. All other embodiments obtained by those of ordinary skill in the art based on the embodiments of the present disclosure without creative efforts should fall within the protection scope of the present disclosure.

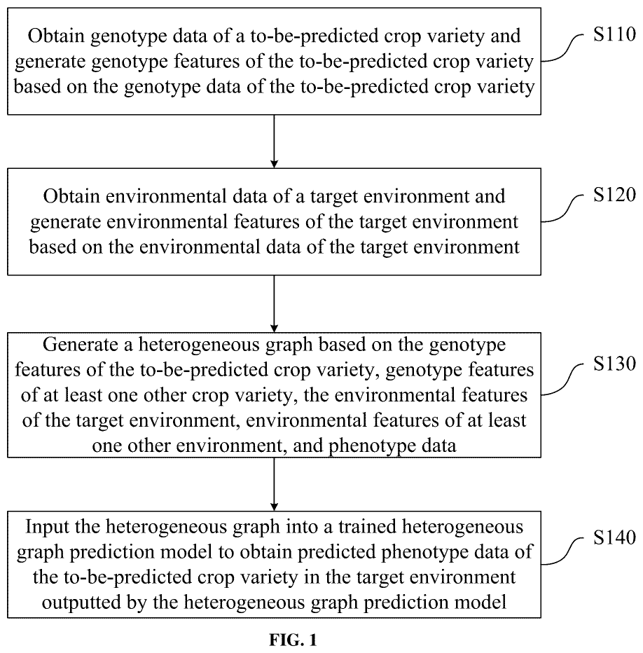

The following describes the genomic prediction method based on a genotype-environment interaction heterogeneous graph provided by the present disclosure in conjunction with FIG. 1. As shown in FIG. 1, the method includes the following steps:
S110: Obtain genotype data of a to-be-predicted crop variety and generate genotype features of the to-be-predicted crop variety based on the genotype data of the to-be-predicted crop variety.
S120: Obtain environmental data of a target environment and generate environmental features of the target environment based on the environmental data of the target environment.
S130: Generate a heterogeneous graph based on the genotype features of the to-be-predicted crop variety, genotype features of at least one other crop variety, the environmental features of the target environment, environmental features of at least one other environment, and phenotype data.
S140: Input the heterogeneous graph into a trained heterogeneous graph prediction model to obtain predicted phenotype data of the to-be-predicted crop variety in the target environment outputted by the heterogeneous graph prediction model.

The heterogeneous graph includes first nodes and second nodes. Each first node corresponds to genotype features of one crop variety, and each second node corresponds to environmental features of one environment; a connecting edge between the first nodes reflects a genetic relationship (represented by similarity) between the crop varieties corresponding to the first nodes, a connecting edge between the second nodes reflects a similarity between the environments corresponding to the second nodes, and a connecting edge between the first node and the second node reflects phenotype of the crop variety corresponding to the first node in the environment corresponding to the second node. The heterogeneous graph prediction model is trained based on a plurality of sets of training data, and each set of training data includes genotype data of a sample crop variety and a phenotype data label of the sample crop variety in a sample environment.

According to the method provided by the present disclosure, genotype features are extracted from genotype data of a to-be-predicted crop variety, environmental features are extracted from environmental data of a target environment, and then a heterogeneous graph is generated based on interrelationships between different crops and different environments, as well as known phenotype data of crops in those environments. A trained heterogeneous graph prediction model is used to process the heterogeneous graph to obtain predicted phenotype data of the to-be-predicted crop variety in the target environment. In this way, the relationship between the genotype of the crop variety and the environment are fully considered during prediction of phenotype data, which can improve the accuracy of phenotype predictions.

The genotype data of the crop variety can include only one type of single-omics data, such as genomic data, or it can include multiple types of single-omics data, such as genomic data, transcriptomic data, and metabolomic data. The environmental data can include data from only one environmental factor, such as meteorological data, or it can include data from multiple environmental factors, such as meteorological data and soil data. Furthermore, the meteorological data may also include indicators such as accumulated temperature, precipitation, and sunshine duration, while the soil environmental data may include indicators such as soil pH and organic matter.

Before the step of generating the genotype features of the to-be-predicted crop variety based on the genotype data of the to-be-predicted crop variety and the step of generating the environmental features of the target environment based on the environmental data of the target environment, raw genotype data and environmental data can be preprocessed, including data alignment and normalization. Specifically, the preprocessing process can include the following steps:
1. Standardize physical locations of genomic data markers, and align the genomic data, where the aligned genomic data is as shown in Table 1.

TABLE 1

| Variety | | | | | | | |
|---|---|---|---|---|---|---|---|
| ID | Gene 1 | Gene 2 | Gene 3 | Gene 4 | Gene 5 | . . . | Gene n |
| V1 | 0 | 1 | 1 | 1 | 0 | . . . | 1 |
| V2 | 1 | 1 | 1 | 0 | 0 | . . . | 1 |
| V3 | 1 | 1 | 0 | 1 | 1 | . . . | 0 |
| . . . | . . . | . . . | . . . | . . . | . . . | . . . | . . . |
| Vn | 0 | 1 | 1 | 1 | 1 | . . . | 0 |

2. Standardize physical locations of transcriptomic data markers, and align the transcriptomic data, where the processed transcriptomic data is as shown in Table 2.

TABLE 2

| Variety | | | | | | | |
|---|---|---|---|---|---|---|---|
| ID | Gene 1 | Gene 2 | Gene 3 | Gene 4 | Gene 5 | . . . | Gene n |
| V1 | 89.88 | 4.51 | 44.68 | 60.70 | 31.97 | . . . | 99.23 |
| V2 | 33.35 | 68.54 | 98.44 | 74.03 | 83.60 | . . . | 15.72 |
| V3 | 82.87 | 10.46 | 66.93 | 21.33 | 64.27 | . . . | 21.26 |
| . . . | . . . | . . . | . . . | . . . | . . . | . . . | . . . |
| Vn | 62.03 | 2.37 | 8.72 | 18.81 | 53.29 | . . . | 84.80 |

3. Standardize metabolite indicators of metabolomic data markers, and align the metabolomic data, where the processed metabolomic data is as shown in Table 3.

TABLE 3

| Variety | Metab- | Metab- | Metab- | Metab- | Metab- | | Metab- |
|---|---|---|---|---|---|---|---|
| ID | olite 1 | olite 2 | olite 3 | olite 4 | olite 5 | . . . | olite n |
| V1 | 7.88 | 9.79 | 4.56 | 7.42 | 8.27 | . . . | 4.12 |
| V2 | 0.9 | 9.8 | 8.41 | 1.71 | 5.31 | . . . | 0.28 |
| V3 | 9.47 | 4.84 | 7.23 | 6.83 | 0.72 | . . . | 0.19 |
| . . . | . . . | . . . | . . . | . . . | . . . | . . . | . . . |
| Vn | 7.79 | 9.54 | 4.87 | 2.01 | 7.84 | . . . | 5.65 |

4. Standardize indicators and units of meteorological and soil environmental data, where the processed environmental data is as shown in Table 4.

TABLE 4

| | Effective | | | | Average |
|---|---|---|---|---|---|
| | Accumulated | Sunshine | | | Wind |
| Environment | Temperature | Hours | Precipitation | | Speed |
| ID | (° C.) | (h) | (mm) | . . . | (m/s) |
| E1 | 2937.25 | 6.91 | 294.91 | . . . | 4.43 |
| E2 | 2840.69 | 7.41 | 431.97 | . . . | 3.74 |
| E3 | 2896.37 | 7.28 | 460.55 | . . . | 5.37 |
| . . . | . . . | . . . | . . . | . . . | . . . |
| E4 | 2695.27 | 5.57 | 313.26 | . . . | 4.32 |

5. Normalize the aligned data. Normalization methods include but are not limited to Z-Score normalization, Max-Min normalization, and standard deviation normalization. For example, using the Z-Score normalization method, the normalization process can be expressed as:

$${x}^{′} = \frac{X - μ}{σ} ,$$

where X is one of the indicators in the variety genomic data, transcriptomic data, metabolomic data, phenotype data, and meteorological and soil environmental data, μ is a mean of the dataset, and σ is a standard deviation.

Generating the genotype features of the to-be-predicted crop variety based on the genotype data of the to-be-predicted crop variety and generating the environmental features of the target environment based on the environmental data of the target environment can involve directly using the genotype data of the to-be-predicted crop variety as the genotype features and the environmental data of the target environment as the environmental features. The method provided by the present disclosure aims to explore the potential associations of multi-omics data, allowing the extracted genotype features and environmental features to provide more information for subsequent phenotype predictions based on the genotype features and environmental features. This is achieved by aggregating the genotype data of the crop variety through a graph neural network to obtain genotype features and aggregating the environmental data of the environment to obtain environmental features. Specifically, the step of generating the genotype features of the to-be-predicted crop variety based on the genotype data of the to-be-predicted crop variety includes:
generating a variety association graph based on the genotype data of the to-be-predicted crop variety and genotype data of a plurality of crop varieties, where each node in the variety association graph corresponds to the genotype data of one crop variety, and a connecting edge between the nodes corresponds to a similarity between the genotype data of the crop varieties; and
inputting the variety association graph into a trained first graph processing model, aggregating genotype data of a first target node with genotype data of other nodes in the variety association graph through the first graph processing model to obtain a first aggregation result, and deriving the genotype features of the to-be-predicted crop variety based on the first aggregation result, where the first target node is a node corresponding to the to-be-predicted crop variety.

The step of generating the environmental features of the target environment based on the environmental data of the target environment includes:
generating an environmental association graph based on the environmental data of the target environment and environmental data of a plurality of environments, where each node in the environmental association graph corresponds to one piece of environmental data, and a connecting edge between the nodes corresponds to a similarity between the environmental data; and
inputting the environmental association graph into a trained second graph processing model, aggregating environmental data of a second target node with environmental data of other nodes in the environmental association graph through the second graph processing model to obtain a second aggregation result, and deriving the environmental features of the target environment based on the second aggregation result, where the second target node is a node corresponding to the target environment.

The first graph processing model, the second graph processing model, and the heterogeneous graph prediction model are jointly trained based on the plurality of sets of training data.

The step of generating the variety association graph based on the genotype data of the to-be-predicted crop variety and the genotype data of the plurality of crop varieties includes:
obtaining first similarities between the genotype data of the to-be-predicted crop variety and the genotype data of other crop varieties;
determining neighboring crop varieties among the other crop varieties based on the first similarities; and
generating the variety association graph based on the genotype data of the to-be-predicted crop variety and the neighboring crop varieties.

The step of generating the environmental association graph based on the environmental data of the target environment and the environmental data of the plurality of environments includes:
obtaining second similarities between the environmental data of the target environment and the environmental data of other environments;
determining neighboring environments among the other environments based on the second similarities; and
generating the environmental association graph based on the environmental data of the target environment and the neighboring environments.

The first similarity can be determined based on a genetic distance (or cosine distance) between genotype data, while the second similarity can be determined based on a cosine distance between environmental data. The formula for calculating the cosine distance between data A and data B is:

$$dist ( A , B ) = 1 - cos ( θ ) = \frac{{‖ A ‖}_{2} {‖ B ‖}_{2} - AB}{{‖ A ‖}_{2} {‖ B ‖}_{2}} .$$

A mean of genetic distances between the genotype data of each pair of varieties is calculated, and N varieties with the smallest distances are selected from varieties with genetic distances to the to-be-predicted crop variety less than the mean to serve as neighboring varieties for the to-be-predicted crop variety. The to-be-predicted crop variety and the neighboring varieties can form a variety association graph. Similarly, a mean of cosine distances between the environmental data of each pair of environments is calculated, and M environments with the smallest distances are selected from environments with cosine distances to the target environment less than the mean to serve as neighboring environments for the target environment. The target environment and the neighboring environments can form an environmental association graph. It can be understood that during generation of the variety association graph/environmental association graph, the calculation is based on one type of genotype data/environmental data. In other words, when the genotype data includes multiple types of single-omics data, a corresponding variety association graph can be generated for each type of single-omics data. When the environmental data includes data from multiple environmental factors, a corresponding environmental association graph can be generated for each type of environmental factor data.

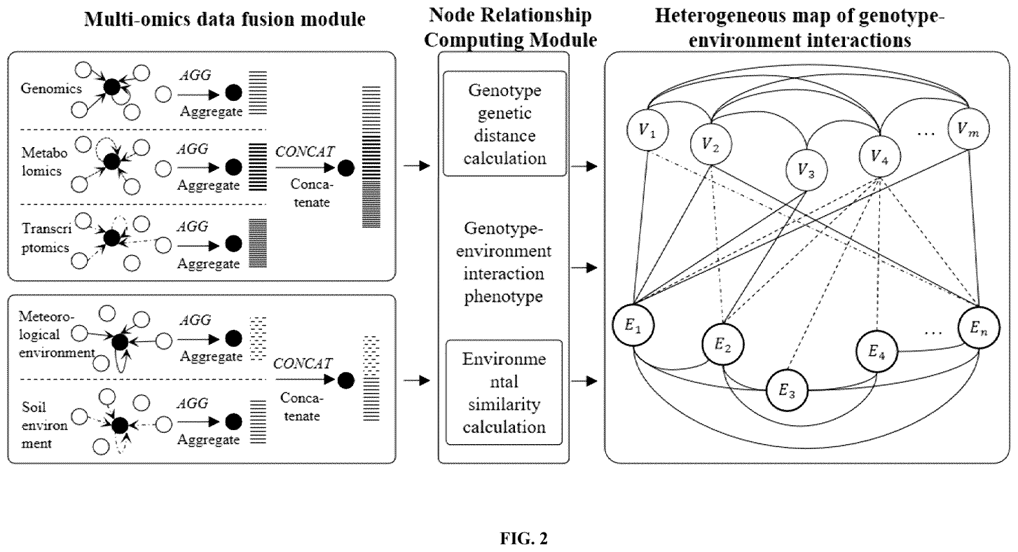

The step of aggregating the genotype data of the first target node with genotype data of other nodes in the variety association graph to obtain the first aggregation result, and the step of aggregating the genotype data of the second target node with the genotype data of other nodes in the environmental association graph to obtain the second aggregation result can be achieved through a graph attention mechanism. As shown in FIG. 2, it can be understood that for each type of single-omics data, a corresponding variety association graph can be generated, thereby obtaining a first aggregation result. Similarly, for each type of environmental data, a corresponding environmental association graph can be generated, thereby obtaining a second aggregation result. Said deriving the genotype features of the to-be-predicted crop variety based on the first aggregation result includes:
obtaining the first aggregation result corresponding to each type of single-omics data, and concatenating all the first aggregation results to obtain the genotype features of the to-be-predicted crop variety.

The environmental data includes multiple types of environmental single-omics data; and said deriving the environmental features of the target environment based on the second aggregation result includes:
obtaining the second aggregation result corresponding to each type of environmental single-omics data, and concatenating all the second aggregation results to obtain the environmental features of the target environment.

Based on the obtained genotype features of the to-be-predicted crop variety and the environmental features of the target environment, the genotype features of other crop varieties and the environmental features of other environments can be correspondingly obtained. A heterogeneous graph is constructed based on the genotype features and the environmental features.

As shown in FIG. 2, the heterogeneous graph includes two types of nodes: first nodes V and second nodes E. The first nodes correspond to crop varieties, and the second nodes correspond to environments. Edge features of connecting edges between the first nodes in the heterogeneous graph reflect the genetic relationships between crop varieties, which can be represented by genetic distances. Edge features of connecting edges between the second nodes reflect the similarity relationships between environments, which can be represented by cosine distances between environmental data or environmental features. Edge features of connecting edges between the first and second nodes reflect phenotype data of the crop varieties in the environments. The phenotype data can include yield per mu or quality indicators. For the phenotype data of multiple varieties, the indicators and units of the phenotype data are standardized in advance, as shown in Table 5.

TABLE 5

| Variety ID | Yield Per Mu (kg) |
|---|---|
| V1 | 726.93 |
| V2 | 659.15 |
| V3 | 769.82 |
| . . . | . . . |
| Vn | 691.37 |

Each node in the heterogeneous graph has two types of features: gene type features and environment type features. For the node corresponding to the crop variety, the gene type feature in the initial node feature is the genotype feature of the crop variety, and the environment type feature is 0. For the node corresponding to the environment, the gene type feature in the initial node feature is 0, and the environment type feature is the environmental feature.

By constructing the heterogeneous graph and performing phenotype prediction based on the heterogeneous graph, the genetic relationships between multiple varieties, the similarity relationships between multiple environments, and the interaction relationships between genotypes and environments can be utilized for phenotype prediction, thereby improving prediction accuracy.

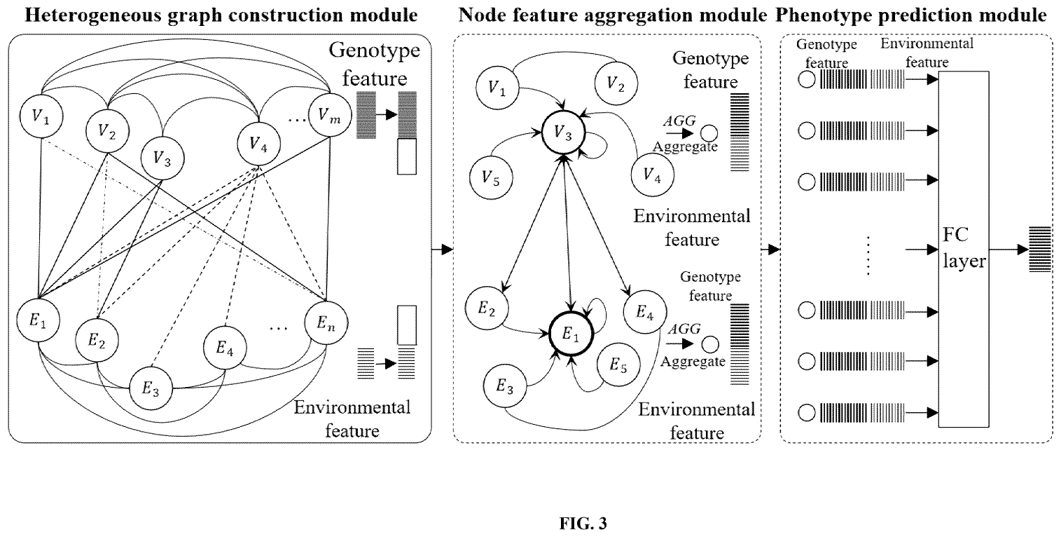

As shown in FIG. 3, the heterogeneous graph prediction model includes a node feature aggregation module and a phenotype prediction module. The heterogeneous graph, to which initial node features and edge features have been added, is input into the heterogenous graph prediction model to obtain the predicted phenotype data of the to-be-predicted crop variety in the target environment outputted by the heterogeneous graph prediction model. This specifically includes:
inputting the heterogeneous graph into the node feature aggregation module, aggregating features of a target first node with features of neighboring nodes in the heterogeneous graph through the node feature aggregation module to obtain a first aggregated feature, and aggregating features of a target second node with features of neighboring nodes to obtain a second aggregated feature, where the target first node is a node corresponding to the to-be-predicted crop variety, and the target second node is a node corresponding to the target environment; and
inputting the first aggregated feature and the second aggregated feature into the phenotype prediction module to obtain the predicted phenotype data of the to-be-predicted crop variety in the target environment outputted by the phenotype prediction module.

The nodes in the heterogeneous graph are divided into two types: isomorphic nodes and heterogeneous nodes. Isomorphic nodes are nodes of the same type. For example, for the target first node, the isomorphic nodes are nodes corresponding to genotypes, while the heterogeneous nodes are nodes corresponding to environments; for the target second node, the isomorphic nodes are nodes corresponding to environments, and the heterogeneous nodes are nodes corresponding to genotypes. In the method provided by the present disclosure, the heterogeneous graph is inputted into the node feature aggregation module, where the node feature aggregation module aggregates the features of the target first node with the features of the neighboring nodes in the heterogeneous graph to obtain the first aggregated feature, and aggregates the features of the target second node with the features of the neighboring nodes to obtain the second aggregated feature. This can fully explore the intrinsic relationship between genotypes and environments, thereby improving the accuracy of the predicted phenotype data of the to-be-predicted crop variety in the target environment.

The process of obtaining the first aggregated feature and the second aggregated feature specifically includes:
by taking the target first node and the target second node as to-be-aggregated nodes, performing the following operations to aggregate the features of the to-be-aggregated nodes with the features of the neighboring nodes:
aggregating the features of the to-be-aggregated node with features of each first neighboring node based on a graph attention mechanism to obtain a first-type feature, where the first neighboring node is the first node among the neighboring nodes of the to-be-aggregated node;
aggregating the features of the to-be-aggregated node with features of each second neighboring node based on the graph attention mechanism to obtain a second-type feature, where the second neighboring node is the second node among the neighboring nodes of the to-be-aggregated node; and
concatenating the first-type feature and the second-type feature.

For the to-be-aggregated node vᵢ, the features of the neighboring nodes are aggregated one by one based on the graph attention mechanism, and an activation function σ is used for transformation. In the attention mechanism, the weight of each neighboring node is αᵢⱼ. The features of the neighboring nodes are aggregated separately based on different types, and the aggregated feature is placed at the position of the corresponding type. In other words, for the target first node (corresponding to the genotype), the gene type features (that is, the genotype features) in the features of its isomorphic neighboring nodes are aggregated to obtain the first-type feature, and the environment type features (that is, the environmental features) in the features of its heterogeneous neighboring nodes are aggregated to obtain the second-type feature.

The formula for the graph attention mechanism can be expressed as:

$${h}_{i}^{t} = σ ( {\sum }_{j ∈ N ( i )} {a}_{ij}^{t} {h}_{j}^{t} ) , ∀ t ∈ Φ ,$$

where σ is the activation function,

$${h}_{j}^{t}$$

represents a t-type feature of node j, N(i) represents the neighboring nodes of node i;

$${a}_{ij}^{t} = soft {max}_{j} ( {e}_{ij}^{t} ) = \frac{exp ( σ ( {e}_{ij}^{t} ) )}{{\sum }_{j ∈ N ( i )} exp ( σ ( {e}_{ij}^{t} ) )} ,$$

where

$${e}_{ij}^{t}$$

represents the degree of importance of the t-type feature of node j to node i, and

$${e}_{ij}^{t} = {att}_{node} ( {h}_{i}^{t} , {h}_{j}^{t} ; Φ ) .$$

αtt_{node} represents a graph operation.

$${h}_{i}^{t}$$

is the first-type feature or second-type feature of the to-be-aggregated node vᵢ (depending on whether t corresponds to the gene type or environment type). In one possible implementation, to optimize the stability of the model, a multi-head attention mechanism is used to aggregate the features of the neighboring nodes for node vᵢ, resulting in a new t-type feature

$${z}_{i}^{t}$$

for node

$${v}_{i} : {z}_{i}^{t} = {\prod }_{k = 1}^{K} σ ( {\sum }_{j ∈ N ( i )} {a}_{ij}^{t} \cdot {h}_{j}^{t} ) ,$$

where K is the number of heads in the multi-head attention mechanism, and

$${z}_{i}^{t}$$

is the first-type feature or second-type feature of the to-be-aggregated node vᵢ (depending on whether t corresponds to the gene type or environment type).

For the first-type feature and second-type feature of node vᵢ, further aggregation is performed to obtain the aggregated feature of node vᵢ. When node vᵢ is the target first node, the aggregated feature is the first aggregated feature; when node vᵢ is the target second node, the aggregated feature is the second aggregated feature.

The aggregation of the first-type feature and second-type feature can be performed through weighted aggregation, represented by the following formula:

$${Z}_{i} = {\sum }_{t ∈ Φ} {β}_{t} {z}_{i}^{t} ,$$

where βₜ is a comprehensive weight for each feature type, and βₜ is a normalized result of

$${w}_{t} : {β}_{t} = \frac{exp ( {w}_{t} )}{{\sum }_{θ ∈ Φ} exp ( {w}_{θ} )} ; {w}_{t} = \frac{1}{\vert V \vert} {\sum }_{i ∈ V} tanh ( W \cdot {z}_{i}^{t} + b ) ,$$

where V is the total number of nodes, W is a convolution matrix parameter, and b is a bias term.

The phenotype prediction module maps the first aggregated feature and the second aggregated feature to one-dimensional predicted phenotype data. As shown in FIG. 3, the phenotype prediction module may include a fully connected (FC) layer.

In one embodiment of the method provided by the present disclosure, in the case where genotype features and environmental features are extracted using the first graph processing model and the second graph processing model, the heterogeneous graph prediction model is jointly trained with the first graph processing model and the second graph processing model. In the case where the first graph processing model and the second graph processing model are not used to extract genotype features and environmental features, the heterogeneous graph prediction model can be trained separately, and the training loss can be obtained by calculating the mean squared error between the predicted results and the labels.

After the predicted phenotype data of the to-be-predicted crop variety in the target environment is obtained, breeding values can be calculated based on the predicted phenotype data, or comparisons can be made with specified control varieties to select superior varieties.

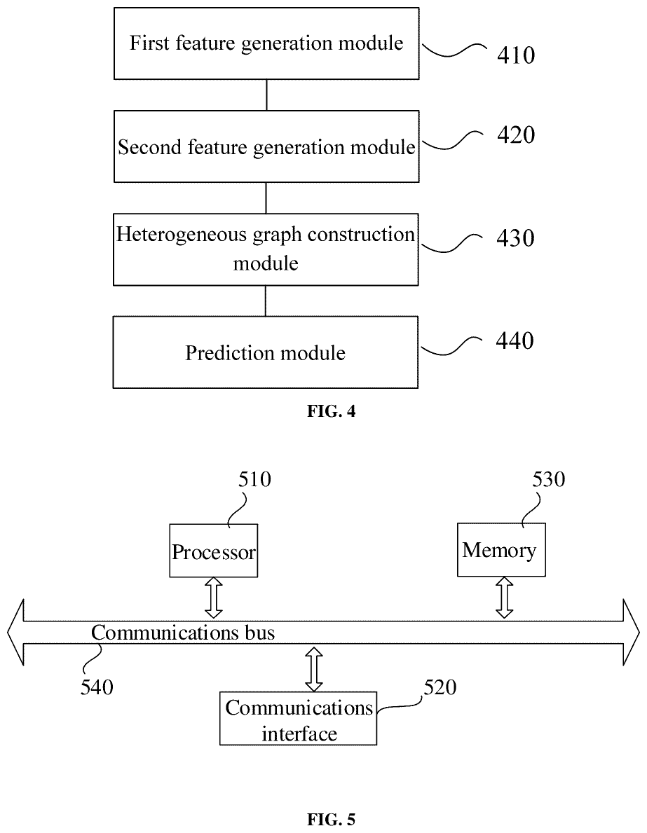

The following describes the apparatus for predicting an environmental phenotype of a crop variety provided by the present disclosure. The apparatus for predicting an environmental phenotype of a crop variety described below corresponds to the genomic prediction method based on a genotype-environment interaction heterogeneous graph described above. As shown in FIG. 4, the apparatus for predicting an environmental phenotype of a crop variety provided by the present disclosure includes:
a first feature generation module 410 configured to obtain genotype data of a to-be-predicted crop variety and generate genotype features of the to-be-predicted crop variety based on the genotype data of the to-be-predicted crop variety;
a second feature generation module 420 configured to obtain environmental data of a target environment and generate environmental features of the target environment based on the environmental data of the target environment;
a heterogeneous graph construction module 430 configured to generate a heterogeneous graph based on the genotype features of the to-be-predicted crop variety, genotype features of at least one other crop variety, the environmental features of the target environment, environmental features of at least one other environment, and phenotype data, where the heterogenous graph includes first nodes and second nodes, each first node corresponds to genotype features of one crop variety, and each second node corresponds to environmental features of one environment; a connecting edge between the first nodes reflects a genetic relationship (represented by similarity) between the crop varieties corresponding to the first nodes, a connecting edge between the second nodes reflects a similarity between the environments corresponding to the second nodes, and a connecting edge between the first node and the second node reflects phenotype of the crop variety corresponding to the first node in the environment corresponding to the second node; and
a prediction module 440 configured to input the heterogeneous graph into a trained heterogeneous graph prediction model to obtain predicted phenotype data of the to-be-predicted crop variety in the target environment outputted by the heterogeneous graph prediction model, where the heterogeneous graph prediction model is trained based on a plurality of sets of training data, each set of training data includes genotype data of a sample crop variety and a phenotype data label of the sample crop variety in a sample environment.

FIG. 5 is a schematic structural diagram of an entity of an electronic device. As shown in FIG. 5, the electronic device may include a processor 510, a communications interface 520, a memory 530, and a communications bus 540. The processor 510, the communications interface 520, and the memory 530 communicate with one another by means of the communications bus 540. The processor 510 can invoke logic instructions in the memory 530 to execute the genomic prediction method based on a genotype-environment interaction heterogenous graph. The method includes: obtaining genotype data of a to-be-predicted crop variety and generating genotype features of the to-be-predicted crop variety based on the genotype data of the to-be-predicted crop variety; obtaining environmental data of a target environment and generating environmental features of the target environment based on the environmental data of the target environment; generating a heterogeneous graph based on the genotype features of the to-be-predicted crop variety, genotype features of at least one other crop variety, the environmental features of the target environment, environmental features of at least one other environment, and phenotype data, where the heterogeneous graph includes first nodes and second nodes, each first node corresponds to genotype features of one crop variety, and each second node corresponds to environmental features of one environment; a connecting edge between the first nodes reflects a genetic relationship (represented by similarity) between the crop varieties corresponding to the first nodes, a connecting edge between the second nodes reflects a similarity between the environments corresponding to the second nodes, and a connecting edge between the first node and the second node reflects phenotype of the crop variety corresponding to the first node in the environment corresponding to the second node; and inputting the heterogenous graph into a trained heterogeneous graph prediction model to obtain predicted phenotype data of the to-be-predicted crop variety in the target environment outputted by the heterogeneous graph prediction model, where the heterogeneous graph prediction model is trained based on a plurality of sets of training data, and each set of training data includes genotype data of a sample crop variety and a phenotype data label of the sample crop variety in a sample environment.

Besides, the logic instructions in the memory 530 may be implemented as a software function unit and be stored in a computer-readable storage medium when sold or used as a separate product. On the basis of such understanding, the technical solutions of the present disclosure essentially or the part contributing to the prior art may be embodied in a form of a software product. The computer software product is stored in a storage medium, and includes several instructions for enabling a computer device (which may be a personal computer, a server, a network device, etc.) to execute all or some steps of the methods described in the embodiments of the present disclosure. The foregoing storage medium includes any medium that can store a program code, such as a universal serial bus (USB) flash disk, a mobile hard disk, a read-only memory (ROM), a random access memory (RAM), a magnetic disk, or an optical disk.

In another aspect, the present disclosure further provides a computer program product. The computer program product includes a computer program stored on a non-transitory computer-readable storage medium. When the computer program is executed by a processor, a computer can execute the foregoing genomic prediction method based on a genotype-environment interaction heterogeneous graph. The method includes: obtaining genotype data of a to-be-predicted crop variety and generating genotype features of the to-be-predicted crop variety based on the genotype data of the to-be-predicted crop variety; obtaining environmental data of a target environment and generating environmental features of the target environment based on the environmental data of the target environment; generating a heterogeneous graph based on the genotype features of the to-be-predicted crop variety, genotype features of at least one other crop variety, the environmental features of the target environment, environmental features of at least one other environment, and phenotype data, where the heterogeneous graph includes first nodes and second nodes, each first node corresponds to genotype features of one crop variety, and each second node corresponds to environmental features of one environment; a connecting edge between the first nodes reflects a genetic relationship (represented by similarity) between the crop varieties corresponding to the first nodes, a connecting edge between the second nodes reflects a similarity between the environments corresponding to the second nodes, and a connecting edge between the first node and the second node reflects phenotype of the crop variety corresponding to the first node in the environment corresponding to the second node; and inputting the heterogeneous graph into a trained heterogeneous graph prediction model to obtain predicted phenotype data of the to-be-predicted crop variety in the target environment outputted by the heterogenous graph prediction model, where the heterogeneous graph prediction model is trained based on a plurality of sets of training data, and each set of training data includes genotype data of a sample crop variety and a phenotype data label of the sample crop variety in a sample environment.

In still another aspect, the present disclosure further provides a non-transitory computer-readable storage medium storing a computer program. The computer program is executed by a processor to implement the foregoing genomic prediction method based on a genotype-environment interaction heterogeneous graph. The method includes: obtaining genotype data of a to-be-predicted crop variety and generating genotype features of the to-be-predicted crop variety based on the genotype data of the to-be-predicted crop variety; obtaining environmental data of a target environment and generating environmental features of the target environment based on the environmental data of the target environment; generating a heterogeneous graph based on the genotype features of the to-be-predicted crop variety, genotype features of at least one other crop variety, the environmental features of the target environment, environmental features of at least one other environment, and phenotype data, where the heterogeneous graph includes first nodes and second nodes, each first node corresponds to genotype features of one crop variety, and each second node corresponds to environmental features of one environment; a connecting edge between the first nodes reflects a genetic relationship (represented by similarity) between the crop varieties corresponding to the first nodes, a connecting edge between the second nodes reflects a similarity between the environments corresponding to the second nodes, and a connecting edge between the first node and the second node reflects phenotype of the crop variety corresponding to the first node in the environment corresponding to the second node; and inputting the heterogeneous graph into a trained heterogeneous graph prediction model to obtain predicted phenotype data of the to-be-predicted crop variety in the target environment outputted by the heterogeneous graph prediction model, where the heterogeneous graph prediction model is trained based on a plurality of sets of training data, and each set of training data includes genotype data of a sample crop variety and a phenotype data label of the sample crop variety in a sample environment.

The apparatus embodiment described above is merely schematic, where the unit described as a separate component may or may not be physically separated, and a component displayed as a unit may or may not be a physical unit, that is, the component may be located at one place, or distributed on multiple network units. Some or all of the modules may be selected based on actual needs to achieve the objectives of the solutions of the embodiments. A person of ordinary skill in the art can understand and implement the embodiments without creative efforts.

Through the description of the foregoing implementations, a person skilled in the art can clearly understand that the implementations can be implemented by means of software plus a necessary universal hardware platform, or certainly, can be implemented by hardware. Based on such understanding, the technical solutions essentially or the part contributing to the prior art may be implemented in a form of a software product. The computer software product may be stored in a computer-readable storage medium such as a ROM/RAM, a magnetic disk, or an optical disk, and includes several instructions for enabling a computer device (which may be a personal computer, a server, a network device, or the like) to execute the methods in the embodiments or parts of the embodiments.

Finally, it should be noted that the foregoing embodiments are only used to illustrate the technical solutions of the present disclosure, and are not intended to limit the present disclosure. Although the present disclosure is described in detail with reference to the foregoing embodiments, those of ordinary skill in the art should understand that they can still modify the technical solutions described in the foregoing embodiments, or make equivalent substitutions to some technical features therein. These modifications or substitutions do not make the essence of the corresponding technical solutions depart from the spirit and scope of the technical solutions in the embodiments of the present disclosure.

1. A method for selecting superior variety based on a genotype-environment interaction heterogeneous graph, the method is performed by an electric device comprising a processor and a memory having stored program instructions, wherein when the processor executes the program instructions stored on the memory, the processor is configured to perform operations, comprising:
a): obtaining genotype data of a target crop variety and generating genotype features of the target crop variety based on the genotype data of the target crop variety;
b): obtaining environmental data of a target environment and generating environmental features of the target environment based on the environmental data of the target environment;
c): generating a heterogeneous graph based on the genotype features of the target crop variety, genotype features of at least one other crop variety, the environmental features of the target environment, environmental features of at least one other environment, and phenotype data, wherein the heterogeneous graph comprises first nodes and second nodes, each first node corresponds to genotype features of one crop variety, and each second node corresponds to environmental features of one environment; a connecting edge between the first nodes reflects a genetic relationship between the crop varieties corresponding to the first nodes, a connecting edge between the second nodes reflects a similarity between the environments corresponding to the second nodes, and a connecting edge between the first node and the second node reflects phenotype data of the crop variety corresponding to the first node in the environment corresponding to the second node;
d): inputting the heterogeneous graph into a trained heterogeneous graph prediction model to obtain predicted phenotype data of the target crop variety in the target environment outputted by the heterogeneous graph prediction model, wherein the heterogeneous graph prediction model is trained based on a plurality of sets of training data, and each set of training data comprises genotype data of a sample crop variety and a phenotype data label of the sample crop variety in a sample environment; and
e): selecting superior varieties by comparing with specified control varieties based on the predicted phenotype data;
wherein said generating the genotype features of the target crop variety based on the genotype data of the target crop variety comprises:
a1): obtaining first similarities between the genotype data of the target crop variety and the genotype data of other crop varieties;
a2): determining neighboring crop varieties among the other crop varieties based on the first similarities; wherein calculating a mean of genetic distances between the genotype data of each pair of varieties, and selecting N varieties with the smallest distances from varieties with genetic distances to the target crop variety less than the mean to serve as neighboring varieties for the target crop variety;
a3): generating the variety association graph based on the genotype data of the target crop variety and the neighboring crop varieties;
wherein each node in the variety association graph corresponds to the genotype data of one crop variety, and a connecting edge between the nodes corresponds to a similarity between the genotype data of the crop varieties; and
a4): inputting the variety association graph into a trained first graph processing model, aggregating genotype data of a first target node with genotype data of other nodes in the variety association graph through the first graph processing model to obtain a first aggregation result, and deriving the genotype features of the target crop variety based on the first aggregation result, wherein the first target node is a node corresponding to the target crop variety;
wherein said generating the environmental features of the target environment based on the environmental data of the target environment comprises:
b1): obtaining second similarities between the environmental data of the target environment and the environmental data of other environments;
b2): determining neighboring environments among the other environments based on the second similarities; wherein calculating a mean of cosine distances between the environmental data of each pair of environments, and selecting M environments with the smallest distances from environments with cosine distances to the target environment less than the mean to serve as neighboring environments for the target environment;
b3): generating the environmental association graph based on the environmental data of the target environment and the neighboring environments;
wherein each node in the environmental association graph corresponds to one type of environmental data, and a connecting edge between the nodes corresponds to a similarity between the environmental data; and
b4): inputting the environmental association graph into a trained second graph processing model, aggregating environmental data of a second target node with environmental data of other nodes in the environmental association graph through the second graph processing model to obtain a second aggregation result, and deriving the environmental features of the target environment based on the second aggregation result, wherein the second target node is a node corresponding to the target environment;
wherein the first graph processing model, the second graph processing model, and the heterogeneous graph prediction model are jointly trained based on the plurality of sets of training data;
wherein determining the first similarities based on a cosine distance between the genotype data, determining the second similarities based on a cosine distance between the environmental data, wherein a formula for calculating the cosine distance between data A and data B is:
$$dist ( A , B ) = 1 - cos ( θ ) = \frac{{‖ A ‖}_{2} {‖ B ‖}_{2} - AB}{{‖ A ‖}_{2} {‖ B ‖}_{2}} .$$

2. The method for selecting superior variety based on the genotype-environment interaction heterogeneous graph according to claim 1, wherein the genotype data comprises a plurality of pieces of genomic single-omics data; and said deriving the genotype features of the target crop variety based on the first aggregation result comprises:
obtaining the first aggregation result corresponding to each piece of genomic single-omics data, and concatenating all the first aggregation results to obtain the genotype features of the target crop variety; and
the environmental data comprises a plurality of pieces of environmental single-omics data; and said deriving the environmental features of the target environment based on the second aggregation result comprises:
obtaining the second aggregation result corresponding to each piece of environmental single-omics data, and concatenating all the second aggregation results to obtain the environmental features of the target environment.

3. The genomic prediction method for selecting superior variety based on the genotype-environment interaction heterogeneous graph according to claim 1, wherein the heterogeneous graph prediction model comprises a node feature aggregation module and a phenotype prediction module; and said inputting the heterogeneous graph into the trained heterogeneous graph prediction model to obtain the predicted phenotype data of the target crop variety in the target environment outputted by the heterogeneous graph prediction model comprises:
inputting the heterogeneous graph into the node feature aggregation module, aggregating features of a target first node with features of neighboring nodes in the heterogeneous graph through the node feature aggregation module to obtain a first aggregated feature, and aggregating features of a target second node with features of neighboring nodes to obtain a second aggregated feature, wherein the target first node is a node corresponding to the target crop variety, and the target second node is a node corresponding to the target environment; and
inputting the first aggregated feature and the second aggregated feature into the phenotype prediction module to obtain the predicted phenotype data of the target crop variety in the target environment outputted by the phenotype prediction module.

4. The method for selecting superior variety based on the genotype-environment interaction heterogeneous graph according to claim 3, wherein said inputting the heterogeneous graph into the node feature aggregation module, aggregating the features of the target first node with the features of the neighboring nodes in the heterogeneous graph through the node feature aggregation module to obtain the first aggregated feature, and aggregating the features of the target second node with the features of the neighboring nodes to obtain the second aggregated feature comprises:
by taking the target first node and the target second node as to-be-aggregated nodes, performing the following operations to aggregate the features of the to-be-aggregated nodes with the features of the neighboring nodes:
aggregating the features of the to-be-aggregated node with features of each first neighboring node based on a graph attention mechanism to obtain a first-type feature, wherein the first neighboring node is the first node among the neighboring nodes of the to-be-aggregated node;
aggregating the features of the to-be-aggregated node with features of each second neighboring node based on the graph attention mechanism to obtain a second-type feature, wherein the second neighboring node is the second node among the neighboring nodes of the to-be-aggregated node; and
concatenating the first-type feature and the second-type feature.

5. An apparatus for predicting an environmental phenotype of a crop variety, comprising:
a first feature generation module configured to obtain genotype data of a to-be-predicted crop variety and generate genotype features of the to-be-predicted crop variety based on the genotype data of the to-be-predicted crop variety;
a second feature generation module configured to obtain environmental data of a target environment and generate environmental features of the target environment based on the environmental data of the target environment;
a heterogeneous graph construction module configured to generate a heterogeneous graph based on the genotype features of the to-be-predicted crop variety, genotype features of at least one other crop variety, the environment features of the target environment, environment features of at least one other environment, and phenotype data, wherein the heterogeneous graph comprises first nodes and second nodes, each first node corresponds to genotype features of one crop variety, and each second node corresponds to environment features of one environment; a connecting edge between the first nodes reflects a genetic relationship between the crop varieties corresponding to the first nodes, a connecting edge between the second nodes reflects a similarity between the environments corresponding to the second nodes, and a connecting edge between the first node and the second node reflects phenotype data of the crop variety corresponding to the first node in the environment corresponding to the second node; and
a prediction module configured to input the heterogeneous graph into a trained heterogeneous graph prediction model to obtain predicted phenotype data of the to-be-predicted crop variety in the target environment outputted by the heterogeneous graph prediction model, wherein the heterogeneous graph prediction model is trained based on a plurality of sets of training data, each set of training data comprises genotype data of a sample crop variety and a phenotype data label of the sample crop variety in a sample environment;
wherein said generating the genotype features of the to-be-predicted crop variety based on the genotype data of the to-be-predicted crop variety comprises:
generating a variety association graph based on the genotype data of the to-be-predicted crop variety and genotype data of a plurality of crop varieties, wherein each node in the variety association graph corresponds to the genotype data of one crop variety, and a connecting edge between the nodes corresponds to a similarity between the genotype data of the crop varieties; and
inputting the variety association graph into a trained first graph processing model, aggregating genotype data of a first target node with genotype data of other nodes in the variety association graph through the first graph processing model to obtain a first aggregation result, and deriving the genotype features of the to-be-predicted crop variety based on the first aggregation result, wherein the first target node is a node corresponding to the to-be-predicted crop variety;
said generating the environmental features of the target environment based on the environmental data of the target environment comprises:
generating an environmental association graph based on the environmental data of the target environment and environmental data of a plurality of environments, wherein each node in the environmental association graph corresponds to one type of environmental data, and a connecting edge between the nodes corresponds to a similarity between the environmental data; and
inputting the environmental association graph into a trained second graph processing model, aggregating environmental data of a second target node with environmental data of other nodes in the environmental association graph through the second graph processing model to obtain a second aggregation result, and deriving the environmental features of the target environment based on the second aggregation result, wherein the second target node is a node corresponding to the target environment;
wherein the first graph processing model, the second graph processing model, and the heterogeneous graph prediction model are jointly trained based on the plurality of sets of training data;
said generating the variety association graph based on the genotype data of the to-be-predicted crop variety and the genotype data of the plurality of crop varieties comprises:
obtaining first similarities between the genotype data of the to-be-predicted crop variety and the genotype data of other crop varieties;
determining neighboring crop varieties among the other crop varieties based on the first similarities; and
generating the variety association graph based on the genotype data of the to-be-predicted crop variety and the neighboring crop varieties;
said generating the environmental association graph based on the environmental data of the target environment and the environmental data of the plurality of environments comprises:
obtaining second similarities between the environmental data of the target environment and the environmental data of other environments;
determining neighboring environments among the other environments based on the second similarities; and
generating the environmental association graph based on the environmental data of the target environment and the neighboring environments.

6. An electronic device, comprising a memory, a processor, and a computer program stored in the memory and executable on the processor, wherein the processor executes the computer program to implement the method for selecting superior variety based on a genotype-environment interaction heterogeneous graph according to claim 1.

7. A non-transitory computer-readable storage medium, storing a computer program, wherein the computer program, when executed by a processor, implements the method for selecting superior variety based on a genotype-environment interaction heterogeneous graph according to claim 1.

8. (canceled)

9. The electronic device according to claim 6, wherein the genotype data comprises a plurality of pieces of genomic single-omics data; and said deriving the genotype features of the target crop variety based on the first aggregation result comprises:
obtaining the first aggregation result corresponding to each piece of genomic single-omics data, and concatenating all the first aggregation results to obtain the genotype features of the target crop variety; and
the environmental data comprises a plurality of pieces of environmental single-omics data; and said deriving the environmental features of the target environment based on the second aggregation result comprises:
obtaining the second aggregation result corresponding to each piece of environmental single-omics data, and concatenating all the second aggregation results to obtain the environmental features of the target environment.

10. The electronic device according to claim 6, wherein the heterogeneous graph prediction model comprises a node feature aggregation module and a phenotype prediction module; and said inputting the heterogeneous graph into the trained heterogeneous graph prediction model to obtain the predicted phenotype data of the target crop variety in the target environment outputted by the heterogeneous graph prediction model comprises:
inputting the heterogeneous graph into the node feature aggregation module, aggregating features of a target first node with features of neighboring nodes in the heterogeneous graph through the node feature aggregation module to obtain a first aggregated feature, and aggregating features of a target second node with features of neighboring nodes to obtain a second aggregated feature, wherein the target first node is a node corresponding to the target crop variety, and the target second node is a node corresponding to the target environment; and
inputting the first aggregated feature and the second aggregated feature into the phenotype prediction module to obtain the predicted phenotype data of the target crop variety in the target environment outputted by the phenotype prediction module.

11. The electronic device according to claim 10, wherein said inputting the heterogeneous graph into the node feature aggregation module, aggregating the features of the target first node with the features of the neighboring nodes in the heterogeneous graph through the node feature aggregation module to obtain the first aggregated feature, and aggregating the features of the target second node with the features of the neighboring nodes to obtain the second aggregated feature comprises:
by taking the target first node and the target second node as to-be-aggregated nodes, performing the following operations to aggregate the features of the to-be-aggregated nodes with the features of the neighboring nodes:
aggregating the features of the to-be-aggregated node with features of each first neighboring node based on a graph attention mechanism to obtain a first-type feature, wherein the first neighboring node is the first node among the neighboring nodes of the to-be-aggregated node;
aggregating the features of the to-be-aggregated node with features of each second neighboring node based on the graph attention mechanism to obtain a second-type feature, wherein the second neighboring node is the second node among the neighboring nodes of the to-be-aggregated node; and
concatenating the first-type feature and the second-type feature.

12. The non-transitory computer-readable storage medium according to claim 7, wherein the genotype data comprises a plurality of pieces of genomic single-omics data; and said deriving the genotype features of the target crop variety based on the first aggregation result comprises:
obtaining the first aggregation result corresponding to each piece of genomic single-omics data, and concatenating all the first aggregation results to obtain the genotype features of the target crop variety; and
the environmental data comprises a plurality of pieces of environmental single-omics data; and said deriving the environmental features of the target environment based on the second aggregation result comprises:
obtaining the second aggregation result corresponding to each piece of environmental single-omics data, and concatenating all the second aggregation results to obtain the environmental features of the target environment.

13. The non-transitory computer-readable storage medium according to claim 7, wherein the heterogeneous graph prediction model comprises a node feature aggregation module and a phenotype prediction module; and said inputting the heterogeneous graph into the trained heterogeneous graph prediction model to obtain the predicted phenotype data of the target crop variety in the target environment outputted by the heterogeneous graph prediction model comprises:
inputting the heterogeneous graph into the node feature aggregation module, aggregating features of a target first node with features of neighboring nodes in the heterogeneous graph through the node feature aggregation module to obtain a first aggregated feature, and aggregating features of a target second node with features of neighboring nodes to obtain a second aggregated feature, wherein the target first node is a node corresponding to the target crop variety, and the target second node is a node corresponding to the target environment; and
inputting the first aggregated feature and the second aggregated feature into the phenotype prediction module to obtain the predicted phenotype data of the target crop variety in the target environment outputted by the phenotype prediction module.

14. The non-transitory computer-readable storage medium according to claim 13, wherein said inputting the heterogeneous graph into the node feature aggregation module, aggregating the features of the target first node with the features of the neighboring nodes in the heterogeneous graph through the node feature aggregation module to obtain the first aggregated feature, and aggregating the features of the target second node with the features of the neighboring nodes to obtain the second aggregated feature comprises:
by taking the target first node and the target second node as to-be-aggregated nodes, performing the following operations to aggregate the features of the to-be-aggregated nodes with the features of the neighboring nodes:
aggregating the features of the to-be-aggregated node with features of each first neighboring node based on a graph attention mechanism to obtain a first-type feature, wherein the first neighboring node is the first node among the neighboring nodes of the to-be-aggregated node;
aggregating the features of the to-be-aggregated node with features of each second neighboring node based on the graph attention mechanism to obtain a second-type feature, wherein the second neighboring node is the second node among the neighboring nodes of the to-be-aggregated node; and
concatenating the first-type feature and the second-type feature.

15. (canceled)

16. (canceled)

17. (canceled)
